(19) United States
(12) Patent Application Publication
(10) Pub. No.: US 2026/0062432 A1
(43) Pub. Date: Mar. 5, 2026

(54) NOVEL BENFOTIAMINE CHOLINE SALT

(71) Applicant: REXPHARMTECH CO., LTD, Gyeonggi-do (KR)

(72) Inventors: Ji Seok YOO, Gyeonggi-do (KR); Yongnam LEE, Gyeonggi-do (KR); Jong Hun LEE, Gyeonggi-do (KR); Ju Young BAEK, Gyeonggi-do (KR); Dae Hyeon KIM, Gyeonggi-do (KR)

(73) Assignee: REXPHARMTECH CO., LTD, Gyeonggi-do (KR)

(21) Appl. No.: 19/108,301

(22) PCT Filed: Sep. 1, 2023
(86) PCT No.: PCT/KR2023/013101

§ 371 Date: Mar. 3, 2025

(30) Foreign Application Priority Data
Sep. 2, 2022 (KR) 10-2022-0111395

(51) Int. Cl. C07F 9/6512; A61K 31/675

(52) U.S. Cl. CPC C07F 9/6512; A61K 31/675; C07B 2200/13

(57) ABSTRACT

The present invention relates to a novel benfotiamine choline salt, a preparation method thereof, and a use thereof. A benfotiamine choline salt according to the present invention has excellent stability and significantly higher water solubility compared to conventional benfotiamine, thus exhibiting improved dissolution and bioavailability. In addition, a benfotiamine choline salt according to the present invention is effective to improve cognitive functions or to prevent or treat cranial nerve diseases, and thus it can be usefully applied as a pharmaceutical raw material or health functional food raw material.

### TECHNICAL FIELD

The present invention relates to a novel benfotiamine choline salt or benfotiamine choline salt crystalline form, a preparation method thereof, and a use thereof. A benfotiamine choline salt or benfotiamine choline salt crystalline form according to the present invention has excellent stability and significantly higher water solubility compared to conventional benfotiamine so that it can exhibit improved dissolution and bioavailability and have effects of improving cognitive functions, or of preventing, ameliorating, or treating degenerative brain diseases.

### BACKGROUND ART

Thiamine is a compound referred to as vitamin B1 having the chemical structure shown below. Since thiamin is a water-soluble vitamin, even when it is administered in a high dose, only an extremely small amount is stored in the body, and all is excreted.

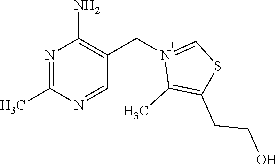

To slow down the excretion rate of thiamine, salts or derivatives have been developed, and representatively, thiamine hydrochloride, thiamine nitrate, fursultiamine, benfotiamine, and bisbentiamine have been developed.

Among these, benfotiamine, which is fat-soluble, reaches the highest concentration in blood about 5 times faster than thiamine hydrochloride or thiamine nitrate, and its bioavailability is about 3.6 times higher.

However, although benfotiamine exhibits fat solubility and thus has an advantage of delaying excretion from the body, it has a problem of exhibiting an extremely low water solubility in water.

Meanwhile, the reason why polymorphism is important in the field of pharmaceuticals is that polymorphism of various forms may exist depending on the drug, and a specific polymorphism often affects the ease of preparation of pharmaceutical raw materials, solubility, storage stability, ease of preparation of finished products, and pharmacological activity in vivo. Therefore, bioavailability may be significantly different between different salts and crystalline forms of a same drug, and with respect to a single drug, some crystalline forms may have biological activity that is higher than that of other crystalline forms.

As described above, studies on polymorphism of drugs have been continuously conducted to secure higher bioactivity and stable raw materials.

### DISCLOSURE

### Technical Problem

One object of the present invention is to provide a benfotiamine choline salt of Formula 1 below:

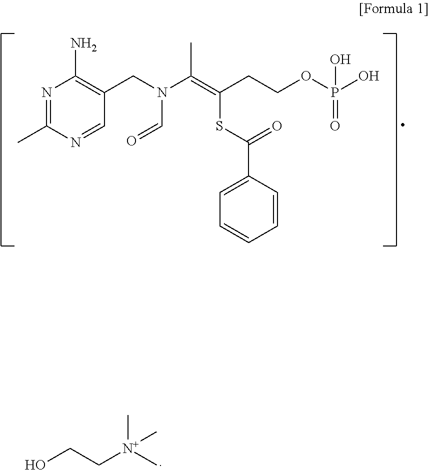

In addition, another object of the present invention is to provide a benfotiamine choline salt crystalline form comprising diffraction peaks at 2θ values of 5.39±0.2°, 8.86±0.2°, 10.59±0.2°, 13.12±0.2°, 15.46±0.2°, 18.11±0.2°, 18.51±0.2°, 22.36±0.2°, 26.16±0.2°, and 30.98±0.2° in powder X-ray diffraction.

Another object of the present invention is to provide a pharmaceutical composition comprising the benfotiamine choline salt or benfotiamine choline salt crystalline form.

In addition, another object of the present invention is to provide a health functional food composition containing the benfotiamine choline salt or benfotiamine choline salt crystalline form.

In addition, another object of the present invention is to provide a method for preparing a benfotiamine choline salt, the method comprising reacting benfotiamine with choline hydroxide.

In addition, another object of the present invention is to provide a method for preparing a benfotiamine choline salt, the method comprising: a first step of adding a lower alcohol having a carbon number of 1 to 4 to an aqueous solution of choline hydroxide and stirring; a second step of adding benfotiamine to the solution of the first step and stirring; a third step of concentrating the solution of the second step under reduced pressure and crystallizing by adding a solvent; and a fourth step of filtering, washing, and drying the solution of the third step.

In addition, another object of the present invention is to provide a method for improving cognitive functions or preventing or treating degenerative brain diseases, the method comprising administering a therapeutically effective amount of a benfotiamine choline salt to a subject in need thereof.

In addition, another object of the present invention is to provide a use of a benfotiamine choline salt for use in preparing a drug for improving cognitive functions or preventing or treating degenerative brain diseases.

### Technical Solution

The present invention provides a benfotiamine choline salt of Formula 1 below:

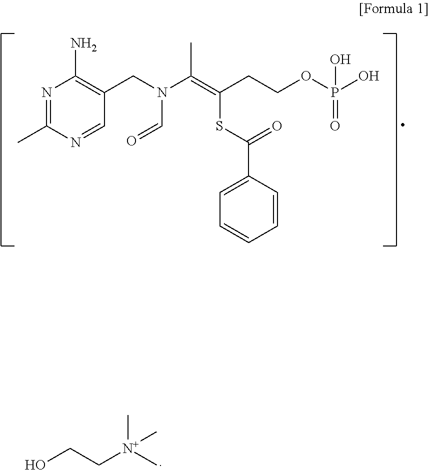

In the present invention, benfotiamine of a structure of Formula 1 above is a thiamine derivative and a fat-soluble S-acyl derivative. Benfotiamine is dephosphorylated to S-benzoylthiamine by ecto-alkaline phosphate present in the intestinal mucosa and hydrolyzed to thiamine by thioesterase in the liver. Benfotiamine has bioavailability that is higher than that of thiamine salts and provides higher levels of thiamine in muscles, brain, liver, and kidneys. Benfotiamine is known to help enhance heart, kidney, nerve, and brain health and prevent aging and complications of diabetes. In particular, it is known that benfotiamine improves spatial memory in a dose-dependent manner and reduces the number of amyloid plaques and the level of phosphorylated tau to improve cognitive functions in a mouse model of Alzheimer's disease (Brain, Volume 133, Issue 5, May 2010, Pages 1342-1351). In addition, benfotiamine has been known to be effective and safe in slowing down the rate of functional decline in patients with mild cognitive impairment or Alzheimer's disease (J Alzheimers Dis, 2020; 78 (3): 989-1010).

Meanwhile, in the present invention, choline in the structure of Formula 1 above is one of water-soluble vitamins and is a type of amino acid that synthesizes lecithin constituting cell membranes in the body and acetylcholine related to memory. Choline plays a role of activating metabolism in vivo and maintaining energy and brain functions. Choline is a precursor to acetylcholine, which is a brain neurotransmitter that plays a pivotal role in memory and learning. Supplementing acetylcholine, which is lacking in patients with brain dysfunctions, normalizes a neurotransmission function that has been lowered due to cranial nerve damage. Patients with cognitive impairment or those with degenerative brain diseases such as Alzheimer's disease lack not only choline but also acetylcholine itself compared to healthy people, and thus the improvement of the patients' symptoms may be expected by increasing the production of acetylcholine through intake.

In the present invention, the term benfotiamine choline salt may refer to not only a compound of Formula 1 above but also a hydrate thereof and an amorphous, partially crystalline, or crystalline form thereof.

Preferably, the benfotiamine choline salt of Formula 1 above of the present invention may be an amorphous, partially crystalline or crystalline compound.

More preferably, the benfotiamine choline salt of Formula 1 above of the present invention is a crystalline form.

Preferably, the benfotiamine choline salt of Formula 1 above of the present invention is characterized in that benfotiamine and choline are bonded at a molar ratio of 1:1.

Preferably, the benfotiamine choline salt of Formula 1 above of the present invention may have 1H nuclear magnetic resonance spectrum (NMR) peaks described below:

¹H NMR (400 MHz, DMSO): 7.91 (s, 1H), 7.81 (s, 1H), 7.69˜7.48 (m, 5H), 6.70 (s, 1H), 4.34 (s, 2H), 3.79 (s, 2H), 3.65 (t, 2H), 3.40 (s, 2H), 3.09 (s, 9H), 2.58 (t, 2H), 2.13 (s, 3H), and 2.11 (s, 3H) ppm.

In addition, preferably, the benfotiamine choline salt of Formula 1 above of the present invention may have a powder X-ray diffraction (PXRD) pattern comprising peaks at 5.39±0.2°, 8.86±0.2°, 10.59±0.2°, 13.12±0.20, 15.46±0.2°, 18.11±0.2°, 18.51±0.2°, 22.36±0.2°, 26.16±0.2°, and 30.98±0.2°.

In addition, preferably, the benfotiamine choline salt of Formula 1 above of the present invention may have a powder X-ray diffraction (PXRD) pattern comprising peaks at 5.39±0.2°, 8.86±0.2°, 9.90±0.2°, 10.59±0.2°, 11.66±0.2°, 13.12±0.2°, 14.01±0.2°, 14.62±0.2°, 15.46±0.2°, 15.91±0.2°, 17.44±0.2°, 18.11±0.2°, 18.51±0.2°, 19.10±0.2°, 19.40±0.2°, 20.48±0.2°, 21.64±0.2°, 22.36±0.2°, 23.36±0.2°, 23.79±0.2°, 24.12±0.2°, 25.37±0.2°, 26.16±0.2°, 26.82±0.2°, 28.18±0.2°, 28.63±0.2°, 29.39±0.2°, 30.27±0.2°, 30.98±0.2°, 31.92±0.2°, 33.25±0.2°, 33.97±0.2°, 35.02±0.2°, 35.75±0.2°, 37.65±0.2°, and 38.42±0.2°.

Preferably, peak positions of a powder X-ray diffraction (PXRD) pattern of a benfotiamine choline salt of the present invention may be substantially identical to FIG. 3.

In addition, present invention provides a the benfotiamine choline salt crystalline form comprising diffraction peaks at 2θ values of 5.39±0.2°, 8.86±0.2°, 10.59±0.2°, 13.12±0.2°, 15.46±0.2°, 18.11±0.2°, 18.51±0.2°, 22.36±0.2°, 26.16±0.2°, and 30.98±0.2°.

Preferably, the benfotiamine choline salt crystalline form may further comprise diffraction peaks at 2θ values of 9.90±0.2°, 11.66±0.2°, 14.01±0.2°, 14.62±0.2°, 15.91±0.2°, 17.44±0.2°, 19.10±0.2°, 19.40±0.2°, 20.48±0.2°, 21.64±0.2°, 23.36±0.2°, 23.79±0.2°, 24.12±0.20, 25.37±0.2°, 26.82±0.2°, 28.18±0.2°, 28.63±0.2°, 29.39±0.2°, 30.27±0.2°, 31.92±0.2°, 33.25±0.2°, 33.97±0.2°, 35.02±0.2°, 35.75±0.2°, 37.65±0.2°, and 38.42±0.2°.

The benfotiamine choline salt or benfotiamine choline salt crystalline form according to the present invention is not simply a compound in the form of a salt or a crystalline form for providing only benfotiamine but a combined compound in which two agonists, benfotiamine and choline, are linked into a single compound, and it can be usefully applied as a pharmaceutical raw material or health functional food raw material for improving cognitive functions or for preventing, ameliorating, or treating cranial nerve diseases such as degenerative brain diseases.

The benfotiamine choline salt or benfotiamine choline salt crystalline form according to the present invention exhibits excellent stability and shows very high water solubility compared to benfotiamine, thereby exhibiting improved bioavailability. In addition, the benfotiamine choline salt or benfotiamine choline salt crystalline form according to the present invention is a single composite compound, and since only a single compound needs to be characterized, handled, and processed, instead of two separate compounds, the preparing and formulating processes can be simplified. Since benfotiamine and choline are combined at a molar ratio of 1:1, there is no problem of uniformity within a formulation, and there is an advantage that they are simultaneously dissolved according to a precise stoichiometric ratio during administration.

In addition, the present invention provides a pharmaceutical composition comprising a benfotiamine choline salt or benfotiamine choline salt crystalline form of Formula 1 above as an active ingredient.

The pharmaceutical composition of the present invention may further comprise a pharmaceutically acceptable additive that is commonly included. The additive may further comprise, for example, any one or more selected from the group consisting of an excipient, a disintegrant, a binder, a lubricant, and a coating agent, and is not limited thereto as long as the desired effects of the present invention are obtained.

The pharmaceutical composition of the present invention may be in a formulation of tablet, capsule, powder, granule, dropping pills, pulvis, bolus, tincture, or poultice, but is not limited thereto. Formulating may be carried out according to any method known in the technical field of the present invention.

The pharmaceutical composition the containing benfotiamine choline salt or benfotiamine choline salt crystalline form of Formula 1 above of the present invention as an active ingredient can be usefully applied as a pharmaceutical composition for improving cognitive functions or preventing or treating degenerative brain diseases. Effects of each of benfotiamine and choline for improving cognitive functions and preventing or treating degenerative brain diseases, and their pharmacological mechanisms are already well known in the art. In addition, in one specific embodiment, a benfotiamine choline salt according to the present invention was treated with hippocampal cells, and the results confirmed that the intracellular levels of p-Akt, p-ERK, and NR2B were effectively increased. Through this, it was confirmed that a benfotiamine choline salt has an effect of improving cognitive functions or memory by helping with Long-Term Potentiation (LPT).

The degenerative brain disease of the present invention may be at least one disease selected from the group consisting of Parkinson's disease, Alzheimer's disease, Huntington's disease, Lou Gehrig's disease, Creutzfeldt-Jakob disease, stroke, multiple sclerosis, learning disabilities, cognitive disorders, neuroinflammation, nerve cell damage, and memory impairment. It may be more than one disease, but is not limited thereto.

In addition, the present invention provides a functional health food composition comprising the benfotiamine choline salt or benfotiamine choline salt crystalline form of Formula 1 above as an active ingredient.

In the present invention, the health functional food composition may be formulated as a conventional health functional food formulation known in the technical field, for example, granule, tablet, capsule, pill, suspension, emulsion, syrup, chewing gum, drink, and the like, but is not limited thereto. The health functional food composition comprising the benfotiamine choline salt or benfotiamine choline salt crystalline form of Formula 1 above of the present invention as an active ingredient can be usefully applied as a health functional food composition for improving cognitive functions or preventing or ameliorating degenerative brain diseases. The effects of benfotiamine and choline for improving cognitive functions and preventing or ameliorating degenerative brain diseases are as described above.

The pharmaceutical composition or health functional food composition of the present invention may further comprise at least one active ingredient exhibiting the same or similar medicinal effect in addition to the benfotiamine choline salt or benfotiamine choline salt crystalline form of Formula 1 above.

In addition, the present invention provides a method for improving cognitive functions or preventing or treating degenerative brain diseases, the method comprising administering a therapeutically effective amount of the benfotiamine choline salt or benfotiamine choline salt crystalline form of Formula 1 above to a subject in need thereof. The subject may be a mammal comprising a human.

The term “therapeutically effective amount” used in the present invention refers to an amount of a benfotiamine choline salt or benfotiamine choline salt crystalline compound that is effective for improving cognitive functions or treating or preventing degenerative brain diseases. Specifically, “therapeutically effective amount” means an amount sufficient to treat a disease with a reasonable benefit/risk ratio that is applicable to medical treatment, and an effective dose level may be determined according to factors comprising the subject type, severity, age, sex, type of disease, activity of drug, sensitivity to drug, administration time, administration route, rate of excretion, treatment duration, and concurrently used drugs and other factors that are well known in the medical field. The pharmaceutical composition of the present invention may be administered as an individual therapeutic agent or in combination with other therapeutic agents, or may be administered sequentially or simultaneously with a commercially available therapeutic agent. In addition, it may be administered by a single-dose administration or multiple-dose administration. It is important to administer an amount such that a maximum effect may be obtained with a minimum amount without side effects in consideration of all the factors described above, and the amount may be easily determined by those skilled in the art. The dose of the pharmaceutical composition of the present invention may be determined by an expert according to various factors such as the patient's conditions, age, sex, and complications. Since an active ingredient of the pharmaceutical composition of the present invention has excellent safety, it can be used even at a dose higher than a determined dose.

In addition, the present invention provides a use of the benfotiamine choline salt or benfotiamine choline salt crystalline compound of Formula 1 above for use in preparing a medicament for improving cognitive functions or preventing or treating degenerative brain diseases. The benfotiamine choline salt or benfotiamine choline salt crystalline form for preparing a medicament may be mixed with pharmaceutically acceptable adjuvants, diluents, carriers, and the like, and may be prepared as a composite formulation in combination with other active agents to have a synergic effect of active ingredients.

In addition, the present invention provides a method for preparing a benfotiamine choline salt, the method comprising reacting benfotiamine with choline hydroxide.

In addition, the present invention provides a method for preparing a benfotiamine choline salt, the method comprising: a first step of adding a lower alcohol having 1 to 4 carbon atoms to an aqueous solution of choline hydroxide and stirring; a second step of adding benfotiamine to the solution of the first step and stirring; a third step of concentrating the solution of the second step under reduced pressure and crystallizing by adding a solvent; and a fourth step of filtering, washing, and drying the solution of the third step.

Details of the preparation methods of the benfotiamine choline salt may be applied as described for the benfotiamine choline salt of the present invention. Specifically, a benfotiamine choline salt is as described above.

In the preparation methods of the benfotiamine choline salt, preferably, benfotiamine and choline hydroxide may be reacted in a 1:1 molar ratio.

In addition, preferably, the lower alcohol having 1 to 4 carbon atoms may be at least one selected from the group consisting of methanol, ethanol, and isopropyl alcohol.

In addition, preferably, the solvent may be at least one selected from the group consisting of acetone, ethyl acetate, and cyclohexane.

More preferably, the solvent may be acetone or ethyl acetate.

Details mentioned in the use, composition, and treatment method of the present invention are equally applied unless contradictory to each other.

### Advantageous Effects

The novel benfotiamine choline salt or benfotiamine choline salt crystalline form of the present invention exhibits improved stability compared to a conventional benfotiamine and has significantly higher water solubility than benfotiamine, which is known to be poorly soluble, so that a drastic increase of dissolution rate and bioavailability is expected. The benfotiamine choline salt or benfotiamine choline salt crystalline form according to the present invention is not simply a compound in the form of a salt or a crystalline form for providing only benfotiamine but a combined compound in which two agonists, benfotiamine and choline, are linked into a single compound, and it has an effect of simultaneously formulating and ingesting benfotiamine and choline. In addition, a benfotiamine choline salt or benfotiamine choline salt crystalline form of the present invention increases the AMPA receptor and strengthens the signals of p-Akt, p-ERK, and NR2B, which are sub-signals, thereby helping with Long-Term Potentiation. Through this, it can be usefully applied as a pharmaceutical raw material for improving cognitive functions or memory or preventing or treating cranial nerve diseases such as degenerative brain diseases.

### DESCRIPTION OF THE DRAWINGS

FIG. 1 shows nuclear magnetic resonance analysis (1H NMR) results of a benfotiamine choline salt prepared in Example 1.

FIG. 2 shows nuclear magnetic resonance analysis (1H NMR) results of benfotiamine, which is Comparative Example.

FIG. 3 shows powder X-ray diffraction analysis results of a benfotiamine choline salt prepared in Example 1.

FIG. 4 is a graph showing solubility test results of a benfotiamine choline salt of Example 1 and benfotiamine, which is Comparative Example.

FIG. 5 shows immunoblot results confirming the degree of activation of neuroplastic factors by treating hippocampal cells with a benfotiamine choline salt of Example 1.

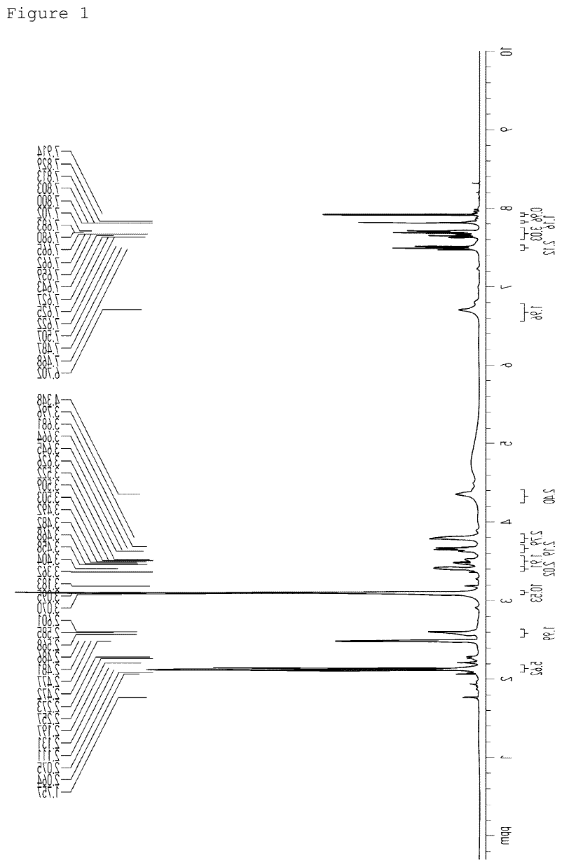

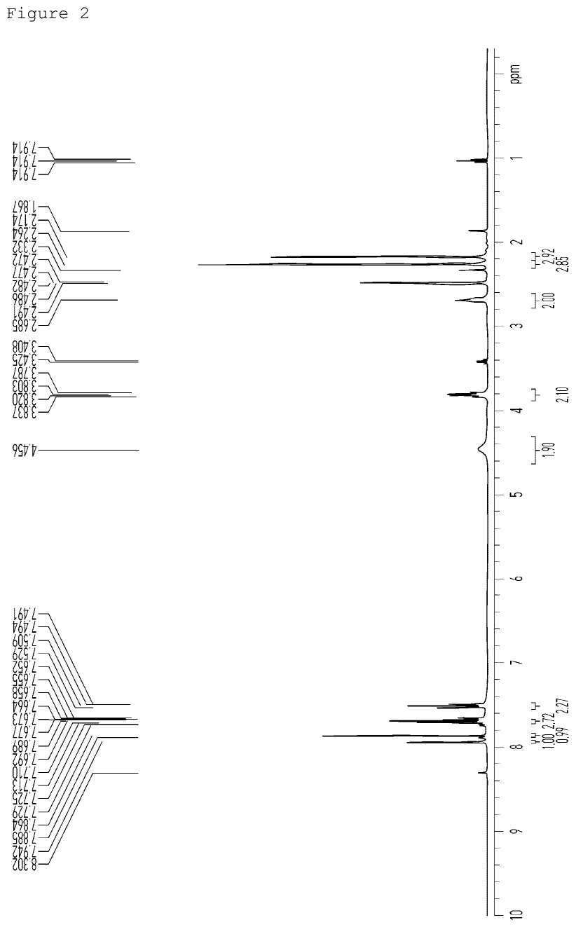

### BEST MODE

Hereinafter, the present invention will be described in detail through examples and experimental examples. However, these examples and experimental examples are provided only for the purpose of illustration to help understand the present invention, and the scope of the present invention is not limited by the examples described below.

### Materials and Apparatuses

In examples, ¹H NMR was performed by using Bruker UltraShield [symbol] 400 (400 MHz), and HPLC was performed by using Agilent 1200 series. Benfotiamine used in examples and comparative examples may be prepared by a known method or commercially purchased, and various reagents and solvents used in a reaction are purchased from Aldrich, TCI, Daejeong, etc.

### Comparative Example: Preparation of Benfotiamine

The benfotiamine of Comparative Example was prepared by purchasing commercially available benfotiamine.

### Example 1: Preparation of Benfotiamine Choline Salt

After dissolving 10 g (36.3 mmol) of a 44% aqueous solution of choline hydroxide in 45 mL of methanol under nitrogen, 17 g (36.3 mmol) of benfotiamine was added thereto and completely dissolved. After concentrating under reduced pressure, 90 mL of acetone was added to crystallize. After salt formation, the resulting mixture was filtered under nitrogen, washed with 45 mL of acetone, and vacuum dried to obtain 18.0 g of benfotiamine choline salt.

¹H NMR (400 MHz, DMSO): 7.91 (s, 1H), 7.81 (s, 1H), 7.69˜7.48 (m, 5H), 6.70 (s, 1H), 4.34 (s, 2H), 3.79 (s, 2H), 3.65 (t, 2H), 3.40 (s, 2H), 3.09 (s, 9H), 2.58 (t, 2H), 2.13 (s, 3H), and 2.11 (s, 3H) ppm.

### Example 2: Preparation of Benfotiamine Choline Salt

After dissolving 10 g (36.3 mmol) of a 44% aqueous solution of choline hydroxide in 150 mL of ethanol under nitrogen, 17 g (36.3 mmol) of benfotiamine was added thereto and completely dissolved. After concentrating under reduced pressure, 90 mL of acetone was added to crystallize. After salt formation, the resulting mixture was filtered under nitrogen, washed with 45 mL of acetone, and vacuum dried to obtain 17.8 g of benfotiamine choline salt.

### Example 3: Preparation of Benfotiamine Choline Salt

After dissolving 10 g (36.3 mmol) of a 44% aqueous solution of choline hydroxide in 45 mL of methanol under nitrogen, 17 g (36.3 mmol) of benfotiamine was added thereto and completely dissolved. After concentrating under reduced pressure, 200 mL of ethyl acetate was added to crystallize. After salt formation, the resulting mixture was filtered under nitrogen, washed with 50 mL of ethyl acetate, and vacuum dried to obtain 18.6 g of benfotiamine choline salt.

### Example 4: Preparation of Benfotiamine Choline Salt

After dissolving 10 g (36.3 mmol) of a 44% aqueous solution of choline hydroxide in 150 mL of ethanol under nitrogen, 17 g (36.3 mmol) of benfotiamine was added thereto and completely dissolved. After concentrating under reduced pressure, 200 mL of ethyl acetate was added to crystallize. After salt formation, the resulting mixture was filtered under nitrogen, washed with 50 mL of ethyl acetate, and vacuum dried to obtain 19.0 g of benfotiamine choline salt.

### Example 5: Preparation of Benfotiamine Choline Salt

After dissolving 10 g (36.3 mmol) of a 44% aqueous solution of choline hydroxide in 45 mL of methanol under nitrogen, 17 g (36.3 mmol) of benfotiamine was added thereto and completely dissolved. After concentrating under reduced pressure, 100 mL of cyclohexane was added to crystallize. After salt formation, the resulting mixture was filtered under nitrogen, washed with 50 mL of cyclohexane, and vacuum dried to obtain 19.5 g of benfotiamine choline salt.

### Example 6: Preparation of Benfotiamine Choline Salt

Under nitrogen, 10 g (36.3 mmol) of a 44% aqueous solution of choline hydroxide and 17 g (36.3 mmol) of benfotiamine were added to 500 mL of isopropyl alcohol and heated to dissolve. The resulting mixture was slowly cooled to room temperature, filtered, washed with 50 mL of isopropyl alcohol, and vacuum dried to obtain 16.7 g of benfotiamine choline salt.

### Experimental Example 1: Stability Test (Accelerated)

A stability test of a pharmaceutical means to confirm the stability of constant quality over time under certain conditions to set the storage method and shelf life of the pharmaceutical. Therefore, an expiration date of a product is set by setting appropriate specifications and evaluating conformance to the specifications by evaluating significant changes based on a prescribed analytical method.

The stability of the benfotiamine choline salt prepared in Example 1 of the present invention and the benfotiamine of Comparative Example was compared.

Specifically, a stability test was conducted under harsh conditions (60° C.±2° C.) according to the ICH guidelines, and an analysis was performed by high-performance liquid chromatography (HPLC) analysis. The results are shown in Table 1.

TABLE 1

| | Initial | Day 3 | Day 7 | Day 14 | Day 21 |
|---|---|---|---|---|---|
| Benfotiamine | 99.3 | 99.2 | 99.1 | 99.0 | 98.2 |
| choline salt | | | | | |
| Benfotiamine | 99.5 | 99.1 | 98.6 | 97.5 | 97.0 |

As shown in Table 1, benfotiamine, which was Comparative Example, showed a purity change of 2.5% as a result of a stability test for 21 days under harsh conditions. On the other hand, the benfotiamine choline salt according to the present invention showed a purity change of 1.1% as a result of the stability test for 21 days under harsh conditions, confirming that the stability was more than double the stability of benfotiamine.

### Experimental Example 2: Solubility Test

To evaluate the solubility of the benfotiamine choline salt according to the present invention, the water solubility of Example 1 and Comparative Example according to the temperature was measured.

Regarding the solubility test method, an experiment was performed by taking 1 mL of purified water and each of solutions at pH 1.2, pH 4.0, and pH 6.8, adding an excessive amount of the benfotiamine choline salt of Example 1 or the benfotiamine of Comparative Example to saturate, ultrasonically extracting for 1 hour, filtering with PVDF, and diluting an appropriate amount.

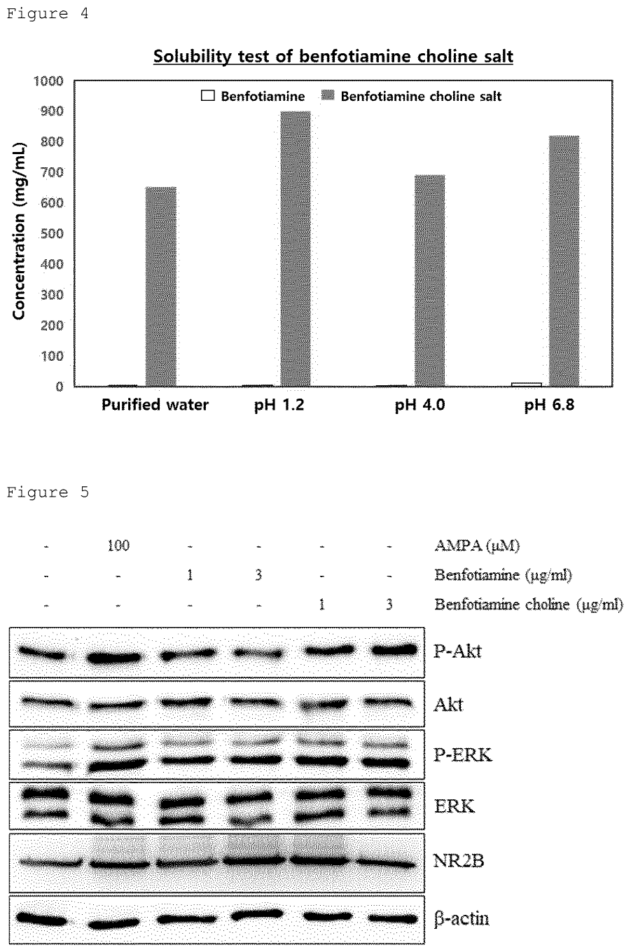

After diluting a reference solution with 50% acetonitrile, a calibration curve (range: 1˜1,000 mg/mL) was prepared by USP compendial HPLC analysis, and the concentration of the saturated solution was obtained from a first-order function of the prepared calibration curve. The results obtained by the solubility test method are shown in Table 2 and FIG. 4 below.

### <HPLC Conditions>

Detector: Ultraviolet spectrophotometer (measurement wavelength 230 nm)
Column: Aegispak C18 (4.6 mm×150 mm, 5 μm) or an equivalent column
Column temperature: constant temperature around 25° C.
Injection volume: 20 μL
Flow rate: 1.0 mL/min
Mobile phase: 0.5% ammonium carbonate solution:methanol mixture (8:2)

TABLE 2

| | Solubility (mg/mL) | |
|---|---|---|
| Dissolution | Comparative Example | Example 1 (benfotiamine |
| conditions | (benfotiamine) | choline salt) |
| Purified water | 3.0 | 652.1 |
| pH 1.2 | 5.6 | 898.4 |
| pH 4.0 | 3.1 | 690.2 |
| pH 6.8 | 6.6 | 820.0 |

As shown in Table 2 and FIG. 4, the benfotiamine of Comparative Example exhibited a low solubility of 3 to 6.6 mg/ml in purified water and solutions at pH 1.2, pH 4.0 and pH 6.8, while the benfotiamine choline salt of Example 1 showed a very high solubility in purified water and all solutions at pH 1.2, pH 4.0, and pH 6.8. Specifically, compared to Comparative Example (benfotiamine), the benfotiamine choline salt of Example 1 exhibited about 124 to 222 times higher solubility. Through this, it was confirmed that by greatly improving the physicochemical properties of benfotiamine, which is a poorly soluble drug, the benfotiamine choline salt of the present invention can drastically increase the absorption and dissolution of the drug.

### Experimental Example 3: PXRD (Powder X-Ray Diffraction) Analysis

In powder X-ray diffractometry, the shape of the diffraction pattern changes when the crystal structure and compound form of materials are different. Using this, the crystal structure of a material may be confirmed in comparison with a reference material. The benfotiamine choline salt according to the present invention and the benfotiamine of Comparative Example as a control were analyzed by using a Rigaku MiniFlex 600.

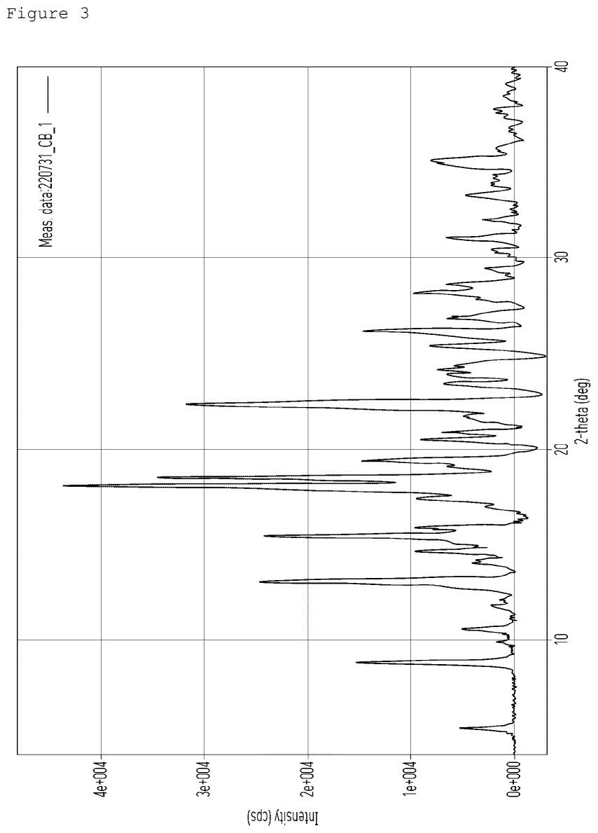

The results showed that, as shown in FIG. 3, it was confirmed that the benfotiamine choline salt of Example 1 of the present invention has characteristic powder X-ray diffraction peak values at 5.39°, 8.86°, 9.90°, 10.59°, 11.66°, 13.12°, 14.01°, 14.62°, 15.46°, 15.91°, 17.44°, 18.11°, 18.51°, 19.10°, 19.40°, 20.48°, 21.64°, 22.36°, 23.36°, 23.79°, 24.12°, 25.37°, 26.16°, 26.82°, 28.18°, 28.63°, 29.39°, 30.27°, 30.98°, 31.92°, 33.25°, 33.97°, 35.02°, 35.75°, 37.65°, and 38.42°.

### Experimental Example 4: Immunoblot Test

### 1. Preparation of Cells for Experiment

All cells used in the experiment were primary cultured hippocampal neurons. Hippocampal neurons were isolated from embryos of SD rats (Sprague-Dawley rats, Samtaco Korea) on gestation Day 18 and Day 19. A hippocampal nerve tissue of fetal rats was physically separated by using a dissecting microscope and then placed in HBSS (Hank's Balanced Salt Solution) supplemented with 0.25% trypsin and treated at 37° C. for 10 minutes. After washing the hippocampal nerve tissue with HBSS, single cells were isolated by carefully pipetting with a pipette. The isolated cells were cultured by filling a 60 mm cell culture dish, coated with Poly-L-lysine (0.5 mg/ml) in advance, with Neurobasal/B27 medium (0.5 mM L-glutamine, 25 μM glutamate, 25 μM 2-mercaptoethanol, 100 U/mL penicillin, 100 μg/ml streptomycin) at 5% CO2 and 37° C. The medium was exchanged on cell culture Day 4 and Day 11, and the cells were used in the experiment on culture Day 14.

### 2. Confirmation of Activation of Neuroplastic Factors in Hippocampal Cells

To confirm p-ERK, p-AKT, and NR2B in cells, the medium of primary cultured hippocampal neurons cultured for 14 days was changed to a B-27-free medium at least 1 hour before drug treatment. As the drug, AMPA, benfotiamine or the benfotiamine choline salt of Example 1 was used, wherein the AMPA was an agonist of AMPA receptor (α-amino-3-hydroxy-5-methyl-4-isoxazoleproionic acid receptor) and was used as a positive control in this experiment.

After treating with 1 or 3 μg/ml of benfotiamine choline salt for 10 minutes, the cells were washed with cold PBS and disrupted on ice. The disrupted cell solution was quantified according to a protein quantification method, and then for protein analysis (immunoblotting), the protein was subjected to electrophoresis on 8% and 10% SDS-polyacrylamide gel. The membrane was subjected to a reaction at room temperature for 1 hour by adding 5% skim milk. A primary antibody was treated with tris buffered saline at a ratio of 1:1000, subjected to a reaction at 4° C. for 24 hours, and then washed three times with TBST (25 mM Tris-Hcl, 140 mM NaCl, 0.1% Tween 20, pH 7.5). After that, a secondary antibody was subjected to a reaction with Anti-Rabbit or Anti-Mouse antibody (HRP-linked) in 3% skim milk at a ratio of 1:5000 for 1 hour. The coupled antibody complex was confirmed by using West Femto Maximum Sensitivity Substrate (Thermo), and the results are shown in FIG. 5.

As shown in FIG. 5, the results confirmed that the benfotiamine choline salt effectively increased the levels of phosphorylated Akt and phosphorylated ERK and NR2B, through which the benfotiamine choline salt has an effect of improving cognitive functions or memory by helping with long-term potentiation (LPT).

1. A benfotiamine choline salt of Formula 1 below:

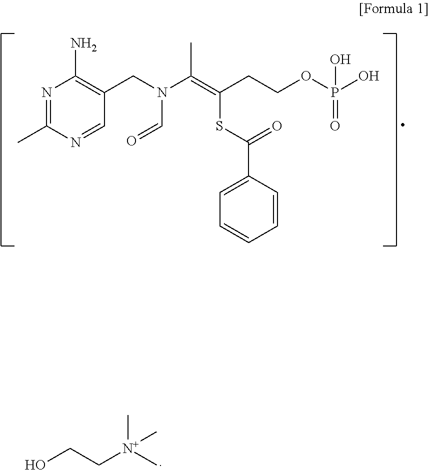

2. The benfotiamine choline salt of claim 1, wherein benfotiamine and choline are bonded at a molar ratio of 1:1.

3. The benfotiamine choline salt of claim 1, wherein the benfotiamine choline salt has 1H nuclear magnetic resonance spectrum peaks described below:
¹H NMR (400 MHz, DMSO): 7.91 (s, 1H), 7.81 (s, 1H), 7.69˜7.48 (m, 5H), 6.70 (s, 1H), 4.34 (s, 2H), 3.79 (s, 2H), 3.65 (t, 2H), 3.40 (s, 2H), 3.09 (s, 9H), 2.58 (t, 2H), 2.13 (s, 3H), and 2.11 (s, 3H) ppm.

4. The benfotiamine choline salt of claim 1, wherein the benfotiamine choline salt of Formula 1 has a powder X-ray diffraction pattern comprising peaks at 5.39±0.2°, 8.86±0.2°, 10.59±0.2°, 13.12±0.2°, 15.46±0.2°, 18.11±0.2°, 18.51±0.2°, 22.36±0.2°, 26.16±0.2°, and 30.98±0.2°.

5. The benfotiamine choline salt of claim 1, wherein the benfotiamine choline salt has a powder X-ray diffraction pattern comprising peaks at 5.39±0.2°, 8.86±0.2°, 9.90±0.2°, 10.59±0.2°, 11.66±0.2°, 13.12±0.2°, 14.01±0.2°, 14.62±0.2°, 15.46±0.2°, 15.91±0.2°, 17.44±0.2°, 18.11±0.2°, 18.51±0.2°, 19.10±0.2°, 19.40±0.2°, 20.48±0.2°, 21.64±0.2°, 22.36±0.2°, 23.36±0.2°, 23.79±0.2°, 24.12±0.2°, 25.37±0.2°, 26.16±0.2°, 26.82±0.2°, 28.18±0.2°, 28.63±0.2°, 29.39±0.2°, 30.27±0.2°, 30.98±0.2°, 31.92±0.2°, 33.25±0.2°, 33.97±0.2°, 35.02±0.2°, 35.75±0.2°, 37.65±0.2°, and 38.42±0.2°.

6. A benfotiamine choline salt crystalline form comprising diffraction peaks at 2θ values of 5.39±0.2°, 8.86±0.2°, 10.59±0.2°, 13.12±0.2°, 15.46±0.2°, 18.11±0.2°, 18.51±0.2°, 22.36±0.2°, 26.16±0.2°, and 30.98±0.2° in powder X-ray diffraction.

7. The benfotiamine choline salt crystalline form of claim 6, further comprising diffraction peaks at 2θ values of 9.90±0.2°, 11.66±0.2°, 14.01±0.2°, 14.62±0.2°, 15.91±0.2°, 17.44±0.2°, 19.10±0.2°, 19.40±0.2°, 20.48±0.2°, 21.64±0.2°, 23.36±0.2°, 23.79±0.2°, 24.12±0.2°, 25.37±0.2°, 26.82±0.2°, 28.18±0.2°, 28.63±0.2°, 29.39±0.2°, 30.27±0.2°, 31.92±0.2°, 33.25±0.2°, 33.97±0.2°, 35.02±0.2°, 35.75±0.2°, 37.65±0.2°, and 38.42±0.2°.

8. A pharmaceutical composition comprising a benfotiamine choline salt of claim 1 as an active ingredient.

9. The pharmaceutical composition of claim 8, wherein the pharmaceutical composition is for improving cognitive functions or preventing or treating degenerative brain diseases.

10. The pharmaceutical composition of claim 8, wherein the pharmaceutical composition is a formulation of tablet, capsule, powder, granule, dropping pills, pulvis, bolus, tincture or poultice.

11. A functional health food composition comprising a benfotiamine choline salt of claim 1 as an active ingredient.

12. The functional health food composition of claim 11, wherein the functional health food composition is for improving cognitive functions or preventing or ameliorating degenerative brain diseases.

13. A pharmaceutical composition comprising a benfotiamine choline salt crystalline form of claim 6 as an active ingredient.

14. The pharmaceutical composition of claim 13, wherein the pharmaceutical composition is for improving cognitive functions or preventing or treating degenerative brain diseases.

15. The pharmaceutical composition according to claim 13, wherein pharmaceutical composition is a formulation of tablet, capsule, powder, granule, dropping pills, pulvis, bolus, tincture or poultice.

16. A functional health food composition comprising a benfotiamine choline salt crystalline form of claim 6 as an active ingredient.

17. The functional health food composition of claim 16, wherein the functional health food composition is for improving cognitive functions or preventing or ameliorating degenerative brain diseases.

18. A method for preparing a benfotiamine choline salt, the method comprising reacting benfotiamine with choline hydroxide.

19. A method for preparing a benfotiamine choline salt, the method comprising:
a first step of adding a lower alcohol having 1 to 4 carbon atoms to an aqueous solution of choline hydroxide and stirring;
a second step of adding benfotiamine to the solution of the first step and stirring;
a third step of concentrating the solution of the second step under reduced pressure and crystallizing by adding a solvent; and
a fourth step of filtering, washing, and drying the solution of the third step.

20. The method for preparing a benfotiamine choline salt of claim 18, wherein the benfotiamine and choline hydroxide are reacted in a molar ratio of 1:1.

21. The method for preparing a benfotiamine choline salt of claim 19, wherein the lower alcohol having 1 to 4 carbon atoms is at least one selected from the group consisting of methanol, ethanol, and isopropyl alcohol.

22. The method for preparing a benfotiamine choline salt of claim 19, wherein the solvent is at least one selected from the group consisting of acetone, ethyl acetate, and cyclohexane.

23. A method for improving cognitive functions or preventing or treating degenerative brain diseases, the method comprising administering a therapeutically effective amount of a benfotiamine choline salt of claim 1 to a subject in need thereof.

24. A use of a benfotiamine choline salt of claim 1 for use in preparing a medicament for improving cognitive functions or preventing or treating degenerative brain diseases.
